(19) United States
(12) Patent Application Publication
(10) Pub. No.: US 2019/0343722 A1
(43) Pub. Date: Nov. 14, 2019

(54) METHOD AND APPARATUS FOR FILLING SYRINGES WITH RETRACTABLE NEEDLE

(71) Applicant: INJEC TECHNOLOGIES INC/LES TECHNOLOGIES INJEC INC., Montreal (CA)

(72) Inventor: Catherine Reisenburg MOLSON, Montreal (CA)

(73) Assignee: INJEC TECHNOLOGIES INC/LES TECHNOLOGIES INJEC INC., Montreal (CA)

(21) Appl. No.: 16/466,925

(22) PCT Filed: Dec. 6, 2017
(86) PCT No.: PCT/CA2017/000263

§ 371 Date: Jun. 5, 2019

Related U.S. Application Data
(60) Provisional application No. 62/430,679, filed on Dec. 6, 2016.

(51) Int. Cl. A61J 1/20; A61M 5/178

(52) U.S. Cl. CPC A61J 1/201; A61M 5/1782; A61M 2005/323; A61J 1/2048; A61J 1/2096

(57) ABSTRACT

A syringe for receiving a fluid from a container. The syringe comprises a barrel for housing the fluid and including an end wall with an opening defined therein, a plunger for slidable engagement within the barrel, and moveable between a retracted position and an extended position, and a needle. The needle has a first end anchored to the plunger and a second distal end, a conduit defined by the needle, and an inlet offset from the second distal end for fluid communication with an opening defined at the second distal end. The plunger includes a plunger seal for engagement within the barrel, the plunger seal defining a passage for receiving the needle. The syringe further comprises an engagement system having a complementary feature to cooperatively engage with a complementary feature of the container to mount the container to the syringe.

### RELATED APPLICATIONS

This application is a national phase of International Application No. PCT/CA2017/000263 filed on Dec. 6, 2017, which claims priority from U.S. Provisional Application No. 62/430,679 filed on Dec. 6, 2016, the entire contents of which are incorporated herein by reference.

### FIELD

The present disclosure relates to a fluid delivery device, and in particular, to a syringe having a retractable needle that can be filled in the field prior to being used.

### BACKGROUND

A conventional syringe may expel a fluid inside its barrel out of an opening, or the barrel may be filled with a fluid received through the opening, by pushing and pulling a plunger of the syringe, respectively. The syringe may have a fitting mounted to the opening such that, for example, a needle may be fixed to the syringe.

Where syringes are fixed with a needle, the sharp exposed tip of the needle may present a risk of accidental nicks or cuts. Further, the exposed needle may be damaged or may be contaminated, thereby affecting the sterility of the syringe, the needle, or contents inside the syringe. Moreover, contaminants, germs, or viruses on the syringe or needle may be transmitted to someone that uses the syringe, is nicked, cut, or otherwise injured by the needle.

In addition, conventional syringes may need to be previously filled with a fluid during their manufacturing. For example, medical syringes may be pre-filled with a medicinal liquid. Accordingly, damage to such syringes may cause the fluid to leak out of the syringe, which may not be recovered or reused. Similarly, if such syringes or needles are contaminated, the liquid may need to be disposed and cannot be recovered or reused.

### SUMMARY

In accordance with an aspect, there is provided a syringe for receiving a fluid from a container, the syringe comprising: a barrel for housing the fluid and including an end wall with an opening defined therein; a plunger for slidable engagement within the barrel, and moveable between a retracted position and an extended position; a needle having: a first end anchored to the plunger and a second distal end; a conduit defined by the needle; and an inlet offset from the second distal end for fluid communication with an opening defined at the second distal end; wherein the plunger includes a plunger seal for engagement within the barrel, the plunger seal defining a passage for receiving the needle; and an engagement system having a complementary feature to cooperatively engage with a complementary feature of the container to mount the container to the syringe.

In some embodiments, the needle is confined in the barrel when the plunger is in the retracted position, and wherein the needle is received in the opening of the end wall when the plunger is in the extended position.

In some embodiments, the syringe comprises a septum that seals the opening of the end wall.

In some embodiments, the syringe comprises a skirt projecting from the end wall and defining a recessed cavity with the end wall.

In some embodiments, the skirt comprises the engagement system to mount the syringe to the container.

In some embodiments, the engagement system comprises a twist and lock feature for mounting the container to the syringe by twisting the container or syringe relative to the other.

In some embodiments, the engagement system comprises a male or female engagement part for mounting the container to the syringe by cooperatively engaging with a complementary male or female engagement part of the container.

In some embodiments, when the plunger is slidably engaged within the barrel and moved from the retracted position to the extended position, the pressure in the barrel is increased.

In some embodiments, when the container is mounted to the syringe and the plunger is in the extended position, the needle provides fluid communication between the container and the barrel for filling the barrel with fluid from the container.

In accordance with another aspect, there is provided a syringe for receiving a fluid from a container, the syringe comprising: a barrel for housing the fluid and including an end wall with an opening defined therein; a plunger for slidable engagement within the barrel, and moveable between a retracted position and an extended position; a needle having: a first end anchored to the plunger and a second distal end; a conduit defined by the needle; and an inlet offset from the second distal end for fluid communication with an opening defined at the second distal end; wherein the plunger includes a plunger seal for engagement within the barrel, the plunger seal defining a passage for receiving the needle; a chamber defined between the plunger and the plunger seal when the plunger and the plunger seal are engaged within the barrel; and an engagement system having a complementary feature to cooperatively engage with a complementary feature of the container to mount the container to the syringe.

In some embodiments, the needle is confined in the barrel when the plunger is in the retracted position, and wherein the needle is received in the opening of the end wall when the plunger is in the extended position.

In some embodiments, the syringe comprises a septum that seals the opening of the end wall.

In some embodiments, the syringe comprises a skirt projecting from the end wall and defining a recessed cavity with the end wall.

In some embodiments, the skirt comprises the engagement system to mount the syringe to the container.

In some embodiments, the engagement system comprises a twist and lock feature for mounting the container to the syringe by twisting the container or syringe relative to the other.

In some embodiments, the engagement system comprises a male or female engagement part for mounting the container to the syringe by cooperatively engaging with a complementary male or female engagement part of the container.

In some embodiments, when the plunger is slidably engaged within the barrel and moved from the retracted position to the extended position, the pressure in the barrel is increased.

In some embodiments, when the container is mounted to the syringe and the plunger is in the extended position, the needle provides fluid communication between the container and the barrel for filling the barrel with fluid from the container.

In some embodiments, a longitudinal length of the skirt is greater than a longitudinal length of the chamber.

In accordance with another aspect, there is provided a syringe for receiving a fluid from a container having a first and second conduit mounted on an adapter of the container, the syringe comprising: a barrel for housing the fluid and including an end wall with an opening defined therein; a plunger for slidable engagement within the barrel, and moveable between a retracted position and an extended position; a needle having: a first end anchored to the plunger and a second distal end; a conduit defined by the needle; and an inlet offset from the second distal end for fluid communication with an opening defined at the second distal end; wherein the plunger includes a plunger seal for engagement within the barrel, the plunger seal defining a passage for receiving the needle; and an engagement system having a complementary feature to cooperatively engage with a complementary feature of the container to mount the container to the syringe; wherein the end wall is configured to receive a first and a second conduit of the container when the container is mounted to the syringe.

Many further features and combinations thereof concerning embodiments described herein will appear to those skilled in the art following a reading of the instant disclosure.

### BRIEF DESCRIPTION OF DRAWINGS

In the figures which illustrate example embodiments:

FIG. 1 is a schematic of a syringe with a retractable needle in a retracted position to be filled by a vial;

FIG. 2 is a schematic of the syringe of FIG. 1 with the needle in an extended position;

FIG. 3 is a schematic of the syringe of FIG. 1 with the needle in a retracted position to be filled by a vial having a bellows device fitted on the vial;

FIG. 4 is a schematic of the syringe of FIG. 3 with the needle in an extended position;

FIG. 5 is a schematic of the syringe of FIG. 1 with the needle in a retracted position to be filled by a vial having two conduits mounted on the vial;

FIG. 6 is a schematic of the syringe of FIG. 5 with the needle in an extended position; and

FIG. 7A to FIG. 7G are schematics of the syringe of FIG. 1 used for injecting a fluid.

### DETAILED DESCRIPTION

As used herein, the terms “proximal”, “back”, or “backwards” refers to positions, directions, or orientations farther from a side of the barrel of the syringe having an end wall from which a tip of a needle extends. The term “distal” or “forward” refers to positions, directions, or orientations closer to the side of the barrel of the syringe having the end wall from which the tip of the needle extends.

There is described and disclosed a syringe with a needle that may be retractable within a barrel of the syringe, a reservoir adapted to receive a fluid, such as a flowable medicinal material, and a plunger to which the needle is anchored. The syringe may have a distal end wall having an external contact surface and a plenum to allow a conduit of rigid construction to penetrate the septum and allow the fluid to pass through to and from the reservoir.

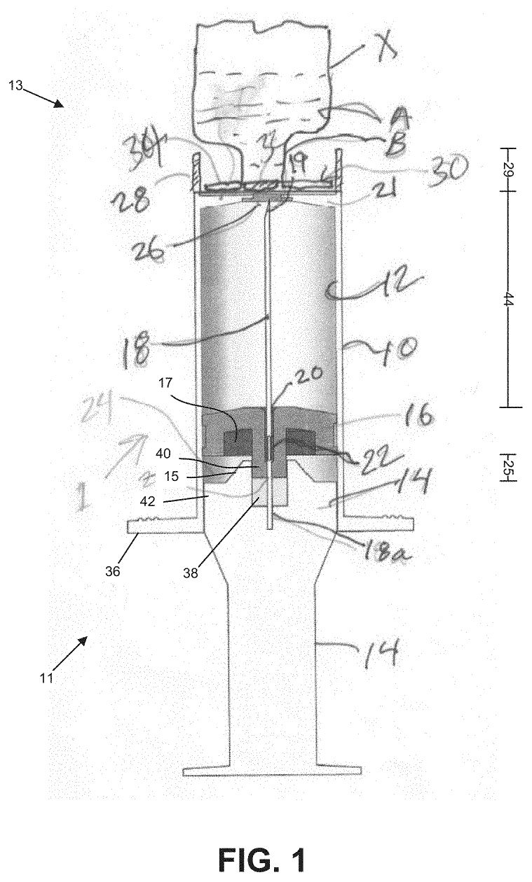

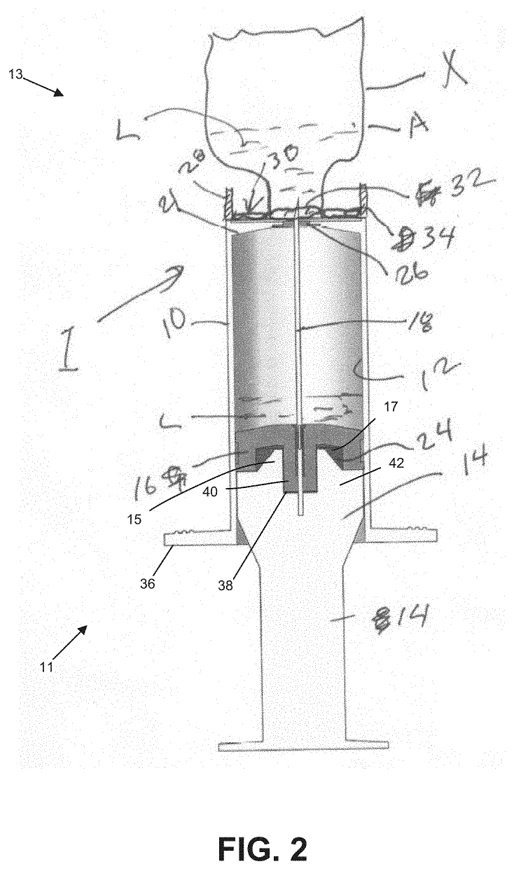

An embodiment of a syringe 1 is shown in FIG. 1 and FIG. 2. The syringe 1 may comprise a barrel 10 that may define a reservoir 12 for containing a fluid. The barrel 10 may define a longitudinal axis along the length of the barrel 10. The barrel 10 may have a generally consistent longitudinal cross-section. For example, the barrel 10 may be generally cylindrical in shape, such that it has a generally circular cross-section. In some embodiments, the barrel 10 may have other geometric cross-sections, such as square, hexagon, octagon, polygon, oval, and the like.

The barrel 10 may have a proximal end 11 and a distal end 13. As depicted in FIG. 1, the proximal end 11 may comprise an opening to receive a plunger 14 and a plunger seal 16. In some embodiments, the barrel 10 may comprise an outer flange 36 that extends outwardly from the barrel 10 at the proximal end 11. In some embodiments, the outer flange 36 may extend radially from the proximal end 11. In some embodiments, the outer flange 36 may be opposing handles. A user using the syringe 1 may securely grip the syringe 1 using the outer flange 36.

At the distal end 13 of the barrel 10, the barrel 10 may comprise a distal end wall 21. The distal end wall 21 may define an opening. The centre of the opening may be aligned with the longitudinal axis of the barrel. In some embodiments, the syringe 1 comprises a septum 26 that may be received or positioned in the opening of the distal end wall 21 to close or seal the opening of the distal end wall 21. The septum 26 may be a septum seal, a sealable septum, a one-way septum, and the like. The septum 26 may be manufactured using rubber or a polymer. In some embodiments, the syringe 1 may comprise a skirt 28 that projects from the distal end wall 21, such as from the periphery of the distal end wall 21, to define a recessed cavity between the distal end wall 21 and the skirt 28. The cross-section of the skirt 28 may be generally similar to the cross-section of the barrel 10. For example, where the barrel 10 is a cylindrical barrel having a generally circular cross-section, the skirt 28 may be an annular skirt. The skirt 28 may have a longitudinal length 29 measured along the longitudinal axis of the barrel 10.

Where the syringe 1 may be filled with a vial containing a fluid, the syringe 1, such as at the skirt 28 or distal end wall 21, and the vial may comprise an engagement system for mounting the vial with the syringe 1, such as at the barrel 10, distal end wall 21, or the skirt 28. The engagement system may have a complementary feature to cooperatively engage with a complementary feature of the container to mount the container to the syringe 1. For example, the engagement system may comprise a male or female engagement part for mounting the container to the syringe 1 by cooperatively engaging with a complementary male or female engagement part of the container. For example, the skirt 28 and the vial may have cooperating male and female engagement parts, such as a rib and channel, a tongue and groove, and the like, for mounting the vial with the syringe 1. The male and female engagement parts may comprise a snap-connection or click-connection feature. As another example, the skirt 28 or the vial may have a clip to clip onto the other of the skirt 28 or the vial to mount the vial with the syringe 1. As another example, the skirt 28 and the vial may have corresponding threading such that the vial may be threaded onto the skirt 28 for engaging the vial with the syringe 1. As another example, the engagement system may have a twist and lock feature such that the vial or syringe 1 may be twisted relative to the other to engage the vial with the syringe 1. An example twist and lock feature may comprise a tab and slot. In some embodiments, the engagement system may be separate from the syringe 1 and the vial and not be part of the syringe 1 or the vial. For example, the engagement system may be a clip or a pin that clips or pins the vial to the syringe 1.

In some embodiments, the syringe 1 and the vial may have corresponding or complementary markings to promote alignment of the syringe 1 and the vial for engaging the vial with the syringe 1, or to indicate that the syringe 1 and the vial are engaged.

The size of the barrel 10 may vary based on the amount of fluid it may contain. For example, the barrel 10 may contain between 0.25 mL to 450 mL of fluid. In some embodiments, the barrel 10 may be marked with volume markings, to indicate the amount of fluid in the barrel 10.

In some embodiments, the barrel 10 may be manufactured using materials such as metal, plastic, glass, and the like, or a combination thereof. Where the barrel 10 is manufactured using plastic, the barrel 10 may be manufactured using blow moulding, injection moulding, plastic forming, 3D printing, and the like.

The syringe 1 may comprise a plunger 14. The plunger 14 may slidably engage within the barrel 10. When the plunger 14 is in slidable engagement with the barrel 10, the plunger 14 may be moveable between a retracted position and an extended position, as described in greater detail herein. The plunger 14 may be dimensioned and sized such that it may be received inside the barrel 10 and fits tightly inside the barrel 10. The barrel 10 and the plunger 14 may be friction fit or interference fit when the plunger 14 is received inside the barrel 10. The interface between the inner surface of the barrel 10 and the plunger 14, when the plunger 14 is inside the barrel 10, is such that the plunger 14 may limit or reduce fluid communication between the reservoir 12 and the external environment of the syringe 1. In some embodiments, the plunger 14 may seal the opening at the proximal end 11 of the barrel 10. That is, if the reservoir 12 contains a liquid, the liquid may not leak out of the barrel 10 through the proximal end 11 if the plunger 14 is received in the barrel 10. There may be sufficient allowance between the barrel 10 and the plunger 14 such that the plunger 14 may independently slide back and forth along the longitudinal axis of the barrel 10. The plunger 14 may define a longitudinal axis. When the plunger 14 is received in the barrel 10, the longitudinal axis of the plunger 14 and the longitudinal axis of the barrel 10 may be generally aligned.

As depicted in FIG. 1, the plunger 14 may comprise a ridge 15. The ridge 15 may be shaped to be received in a recess 17 of a plunger seal 16 of the syringe 1. In some embodiments, the ridge 15 may be an annular ridge that extends around the longitudinal axis of the plunger 14. The plunger 14 may also define a cavity 38. The cavity 38 may be shaped to receive a plunger seal extension 40 of a plunger seal 16.

In some embodiments, the plunger 14 may be manufactured using materials such as plastic, and may be manufactured using blow moulding, injection moulding, plastic forming, 3D printing, and the like. The head 42 of the plunger 14 may be manufactured with rubber.

The syringe 1 may comprise a plunger seal 16. The plunger 14 may include the plunger seal 16 (e.g. at the head or distal end of the plunger 14). In some embodiments, as depicted in FIG. 1, the plunger 14 and the plunger seal 16 may be separate. The plunger seal 16 may engage within the barrel 10. The plunger seal 16 may be dimensioned and sized such that it may be received inside the barrel 10 and fits tightly inside the barrel 10. The barrel 10 and the plunger seal 16 may be friction fit or interference fit when the plunger seal 16 is received inside the barrel 10. The interface between the inner surface of the barrel 10 and the plunger seal 16, when the plunger seal 16 is inside the barrel 10, may be such that the plunger seal 16 may limit or reduce fluid communication across the interface between the inner surface of the barrel 10 and the plunger seal 16. The plunger seal 16 may independently slide back and forth along the longitudinal axis of the barrel 10. The plunger seal 16 may define a longitudinal axis, and when the plunger seal 16 is received in the barrel 10, the longitudinal axis of the plunger seal 16 and the longitudinal axis of the barrel 10 may be generally aligned. The plunger seal 16 may define a passage 20 that extends into or through the plunger seal 16. The passage 20 may extend generally along the longitudinal axis of the plunger seal 16.

The passage 20 may be configured to receive a needle 18 of the syringe 1. As depicted in FIG. 1, at least a portion of the passage 20 may have a diameter sufficiently large so as to allow a fluid to flow in the space so formed. In some embodiments, a first portion of the passage 20 may have a first diameter, and a second portion of the passage 20 may have a second diameter that is shorter than the first diameter, such that the second portion of the passage 20 may be narrower than the first portion of the passage 20. In some embodiments, the first portion of the passage 20 may be a distal portion of the passage 20, and the second portion of the passage 20 may be a proximal portion of the passage 20. As depicted in FIG. 1, the distal portion of the passage 20 may have a diameter sufficiently large so as to allow a fluid to flow in the space so formed. In some embodiments, the passage 20 may align the needle 18 generally along the longitudinal axis of the barrel 10 when the needle 18 is received in the barrel 10. In some embodiments, a narrower portion of the passage 20 may contact the needle 18 to align the needle 18 generally along the longitudinal axis of the barrel 10. The interface between the needle 18 and a portion of the passage 20 that may contact the needle 18 may provide limited or reduced fluid communication through said interface. In some embodiments, when the needle 18 is received in the passage 20 of the plunger seal 16, there may be limited or reduced fluid communication through the passage 20.

As depicted in FIG. 1, the plunger seal 16 may define a recess 17 configured to receive the ridge 15 of the plunger 14. In some embodiments, the recess 17 may be an annular recess that extends around the longitudinal axis of the plunger seal 16. The plunger seal 16 may also comprise the plunger seal extension 40. The plunger seal extension 40 may be shaped to be received in the cavity 38 of the plunger 14.

Similar to the plunger 14, the plunger seal 16 may be manufactured using plastic, rubber, or both. The plunger seal 16 may be manufactured using blow moulding, injection moulding, plastic forming, 3D printing, and the like.

As depicted in FIG. 1, the plunger seal 16 and the plunger 14 may be received in the barrel 10 of the syringe 1. The plunger seal 16 may be positioned at an intermediate position between the proximal end 11 and distal end 13 of the barrel 10, and the plunger 14 may be positioned at the proximal end 11 of the barrel 10. In some embodiments, the ridge 15 and cavity 38 of the plunger 14, and the recess 17 and plunger seal extension 40 of the plunger seal 16, may be cooperatively configured such that, when the plunger 14 is pushed towards the plunger seal 16, the ridge 15 may be received in the recess 17, and the plunger seal extension 40 may be received in the cavity 38.

The syringe 1 may comprise a needle 18. In some embodiments, the needle 18 is a generally straight tube manufactured using metal. The needle 18 has a first end and a second end. The needle 18 has a proximal end (e.g. the first end) and a distal end (e.g. the second end). The proximal end of the needle 18 may be anchored or mounted in a head 42 of the plunger 14, at 18a. When the needle 18 is mounted to the plunger 14, the needle 18 projects from the plunger 14 along a longitudinal axis of the plunger 14. The distal end of the needle 18 may be a free end or an open end having a beveled tip 19. When the plunger 14 and the plunger seal 16 are received in the barrel 10, the needle 18 may extend through the passage 20 of the plunger seal 16. When the needle 18 is received in the barrel 10, the needle 18 may be aligned with the longitudinal axis of the barrel 10.

The needle 18 may define an internal conduit that extends generally along the needle 18. The distal end of the internal conduit may be a free end or open end having the beveled tip 19. The opening may be defined at the distal end (e.g. second end) of the needle 18. The proximal end of the internal conduit may not be a free end or open end. In some embodiments, the needle 18 comprises an inlet 22, such as a lateral inlet as illustrated in FIG. 1, which communicates with the space defined by the passage 20. The inlet 22 may be offset from the second distal end of the needle 18. As depicted in FIG. 1, the inlet 22 is positioned near the proximal end (e.g. first end) of the needle 18. The inlet 22 may extend transversally across a portion of the needle 18, and may allow for fluid communication from outside the needle 18 into the internal conduit of the needle 18. The inlet 22 may allow for fluid with the opening defined at the second distal end. Where the needle 18 is mounted to the plunger 14 and received in the passage 20 of the plunger 16, the inlet 22 may allow fluid communication between the passage 20 and the internal conduit inside the needle 18, such that fluid may flow from the barrel through the inlet 22 to the tip 19 of the needle 18.

The diameter and length of the needle 18 may vary based on the type of fluid and the amount of fluid to be contained by the barrel 10. For example, the diameter of the needle 18 may range between a 6 gauge needle to a 34 gauge needle. As another example, the length of the needle may range from ⅜″ to 3-½″.

The needle 18 may be manufactured using a material that may be biocompatible, pharmacologically inert, sterilisable, or non-toxic. For example, the needle 18 may be manufactured using stainless steel or carbon steel. To prevent corrosion, the needle 18 may be coated with nickel. The needle 18 may be manufactured by tube drawing and the end 19 may be beveled to create the sharp point, may be die cast, and the like.

FIG. 1 illustrates the barrel 10, plunger 14, plunger seal 16, and needle 18 assembled together to form the syringe 1. As depicted in FIG. 1, the plunger seal 16 may be positioned at an intermediate position along the length of the barrel 10 between the proximal end 11 and the distal end 13 of the barrel 10, and the head 42 of the plunger 14 may be positioned near the proximal end 11 of the barrel 10. A chamber 24 may be defined between the plunger 14 and the plunger seal 16 when the plunger 14 and the plunger seal 16 are engaged within the barrel. The chamber 24 may have a longitudinal length 25 measured along the longitudinal axis of the barrel 10. In some embodiments, the longitudinal length 25 of the chamber 24 may be shorter than the longitudinal length 29 of the skirt 28. The reservoir 12 may be defined between the plunger seal 16, the peripheral wall of the barrel 10, and the distal end wall 21.

FIG. 1 illustrates the syringe 1 in its initial configuration, prior to filling. Prior to filling, the fluid in the barrel 10 of the syringe 1 may be air. Accordingly, the fluid in the reservoir 12 and the chamber 24 may be air. Prior to filling, the needle 18 may be confined in the barrel 10 in a retracted position, such that the beveled tip 19 has not pierced the septum 26 and not exposed outside the barrel 10.

When the needle 18 is in the retracted position, as depicted in FIG. 1, the plunger seal 16 may surround the inlet 22. Even though the plunger seal 16 may surround the inlet 22, at least a portion of the passage 20 may be sufficiently large to define a recess such that the reservoir 12 and the internal conduit of the needle 18 may be in fluid communication via the inlet 22.

The syringe 1 may be filled with a liquid, which may be stored in a container, such as a vial X. The vial X may contain a fluid, such as a liquid L, which may be a medicinal liquid. The vial X includes a vessel A, having a neck B. The vial X may be initially sealed with a removable cap, cover or seal (not shown), such that the liquid may not leak out of the vessel A. The removable cap, cover, or seal may be a vacuum seal that seals the vial X. To draw liquid out of the vial X, the vial X may need to fit within the skirt 28 and engage the distal end wall 21, and may be similarly sized and dimensioned as the skirt 28 and the distal end wall 21. Accordingly, the removable cap, cover, or seal of the vial X may be replaced by a filling adapter 30 that may include a sealing panel 34, which may be adapted to sealingly engage the vial X against the distal end wall 21 and the skirt. The sealing panel 34 may be sized and dimensioned to be similar to the size and dimensions of the distal end wall 21 and the skirt 28 such that the filling adapter 30 may sealingly engage the distal end wall 21 and the skirt 28. For example, where the distal end wall 21 is a generally flat wall, the sealing panel 34 may also be generally flat. As another example, where the barrel 10 is a generally cylindrical barrel 10 and the distal end wall 21 is generally circular and the skirt 28 has a generally circular cross-section, the sealing panel 34 may have a generally circular periphery having a diameter that may be generally the same as the inner diameter of the skirt 28 of the syringe 1. At the center of the filling adapter 30 may be a septum 32. When the vial X is sealing engaged with the distal end wall 21 and skirt 28 of the syringe 1, the septum 32 of the vial X may be coincident with the septum 26 of the syringe 1 or may overlap the septum 26 of the syringe 1, or the septum 26 of the syringe 1 may overlap the septum 32 of the vial X. The vial X may be placed against the distal end wall 21 of the syringe 1 and end to end with the distal end wall 21 of the syringe 1. The skirt 28 may allow for proper alignment of the septum 26 of the syringe 1 and the septum 32 of the vial X.

When the liquid is to be transferred from the vial X to the syringe 1, the vial X may be received in the recessed cavity defined by the distal end wall 21 and the skirt 28 and in sealing engagement with the distal end wall 21 and the skirt 28, as depicted in FIG. 1 and FIG. 2. Then, a force is applied to the plunger 14 and the plunger 14 is pressed forward, as shown in FIG. 2, a distance corresponding to the longitudinal length 25 of the chamber 24. As the plunger 14 moves forward by the longitudinal length 25 of the chamber 24, the needle 18 mounted on the plunger 14 also moves forward by the longitudinal length 25 of the chamber 24. This permits the tip 19 of the needle 18 to pierce the septum 26 of the syringe 1 and the septum 32 of the vial X, and a portion of the needle 18 is inserted into the vial X. Accordingly, the needle 18 may be moved from a retracted position, as depicted in FIG. 1, to an extended position, as depicted in FIG. 2.

When the plunger 14 is in the extended position, the needle 18 may be received in the opening defined by the distal end wall 21. Where the opening defined by the distal end wall 21 is closed or sealed with the septum 26, the needle may pierce the septum 26. When the needle 18 pierces the septum 26, the septum 26 is sealingly engaged with the needle 18 to reduce or limit fluid communication through the opening of the distal end wall 21.

When the needle 18 is in the extended position, as depicted in FIG. 2, the plunger seal 16 may surround the inlet 22. Even though the plunger seal 16 may surround the inlet 22, at least a portion of the passage 20 may be sufficiently large to define a recess such that the reservoir 12 and the internal conduit of the needle 18 may be in fluid communication via the inlet 22.

The plunger 14 may be moved forward until the plunger 14 abuts the plunger seal 16. When the plunger 14 abuts the plunger seal 16, the ridge 15 of the plunger 14 may be received in the recess 17 of the plunger seal 16, and the plunger seal extension 40 of the plunger seal 16 is received in the cavity 38 of the plunger 14.

In some embodiments, when the container is mounted to the syringe 1 and the plunger 14 is in the extended position, the needle 18 may provide fluid communication between the container and the barrel 10 for filling the barrel 10 with fluid from the container.

The movement of the plunger 14 pressurizes the air in the barrel 10, such as the air in the reservoir 12 or chamber 24, such that the pressurized air flows through the inlet 22 of the needle 18 and the internal conduit of the needle 18, out of the tip 19, and into the vessel A. The pressurized air displaces the liquid from the vial X by gravity into the needle 18. Once the pressure in the reservoir 12 and the vessel A have reached equilibrium, the liquid may gradually flow into the reservoir 12 by flowing through the tip 19, the internal conduit of needle 18, the inlet 22 and passage 20, and into the reservoir 12. Accordingly, upon piercing the vial X, the liquid in the vial X may flow from vessel A to the reservoir 12. The liquid in the vial X may flow from the vessel A to the reservoir 12 until the liquid level in the vessel A is below the longitudinal length of the needle 18 that is inserted in the vessel A.

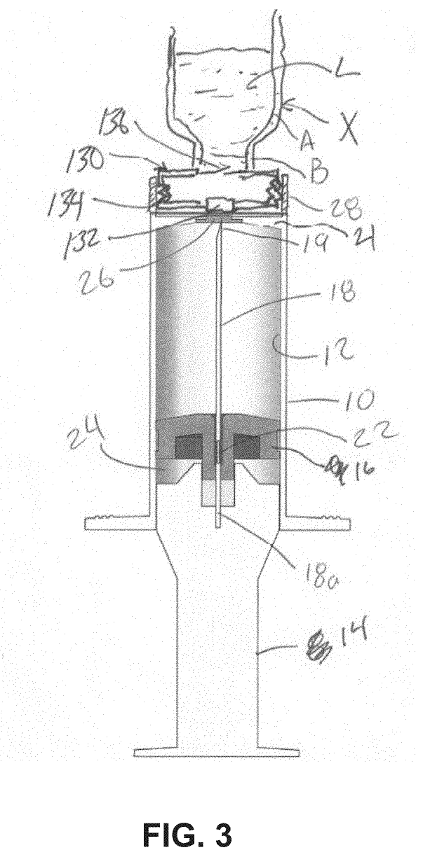

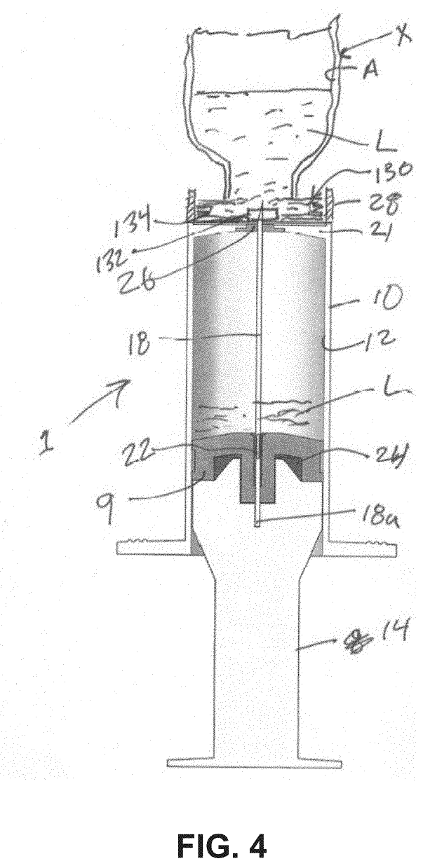

Another embodiment is illustrated in FIGS. 3 and 4. In this embodiment, the filling adapter 30 may be a bellows device 130 that may be fitted to the vial X after the bellows device 130 has replaced the removable cap, cover or seal initially covering the vial X. The bellows 130 may comprise a septum 132 at a proximal end wall of the bellows 130 and a valve 136 at a distal end wall of the bellows 130. The septum 132 may oppose the valve 136, as depicted in FIG. 3. The bellows 130 may comprise a corrugated side wall 134. The bellows 130 may contain a fluid, such as air. When the reservoir 12 of the syringe 1 is to be filled, the bellows device 130 may be fitted at or to the neck B of the vial X. Then, the vial X may be received in the recessed cavity defined by the skirt 28 and the distal end wall 21, such that the bellows 130 may be located within the recessed cavity defined by the skirt 28 and the distal end wall 21, and the bellows 130 may be engaged with the skirt 28 and the distal wall 21. The dimensions of the bellows 130 may be such that, when the bellows 130 is received in the recessed cavity defined by the distal end wall 21 and the skirt 28 of the syringe 1, the septum 132 of the bellows 130 may be coincident with the septum 26 of the syringe 1 or may overlap the septum 26 of the syringe 1, or the septum 26 of the syringe 1 may overlap the septum 132 of the bellows 130. For the liquid in the vial X to flow into the reservoir 12, as illustrated in FIG. 4, the vial X may be pressed in the direction of the distal end wall 21, thereby compressing the bellows 130 and causing an increased in pressure within the chamber of the bellows 130 to open the valve 136. The compression of the bellows 130 may force compressed fluid, such as compressed air, through the opened valve 136 and into the vessel A, and increase the pressure in the vessel A. While the compressed fluid from the bellows 130 is flowing into the vessel A, the plunger 14 is moved in a forward direction until the plunger 14 abuts the plunger seal 16, so that the air in the barrel 10, such as the air in the chamber 24 or the reservoir 12, is compressed. The compressed air may flow through the opening 22 of the needle 18 out of the tip 19, while the tip 19 of the needle 18 pierces the septum 26 and septum 132. The increased pressure in the vessel A may initiate the flow of liquid L from the vessel A through the conduit formed by the needle 18 ultimately into the reservoir 12. Accordingly, upon piercing the vial X, the liquid in the vial X may flow from vessel A to the reservoir 12.

In some embodiments, the vial X illustrated in FIG. 3 and FIG. 4 where the filling adapter 30 is the bellows device 130, the syringe 1 and the vial X may have an engagement feature that has a twist and lock feature, such that when the vial X is pushed down to compress the bellows device 130, the vial X may be twisted to engage the vial X with the syringe 1 and lock the syringe X with the syringe 1. In some embodiments, when twisting the vial X to engage the vial X with the syringe 1, the twisting may cause the compression of the bellows device 130.

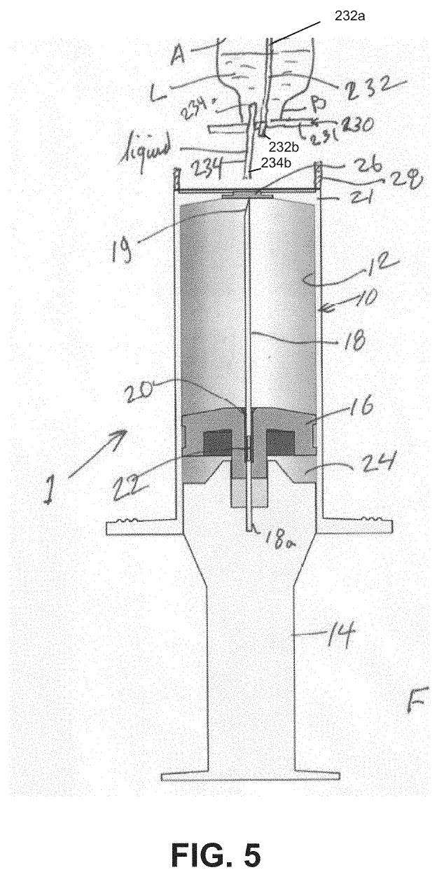

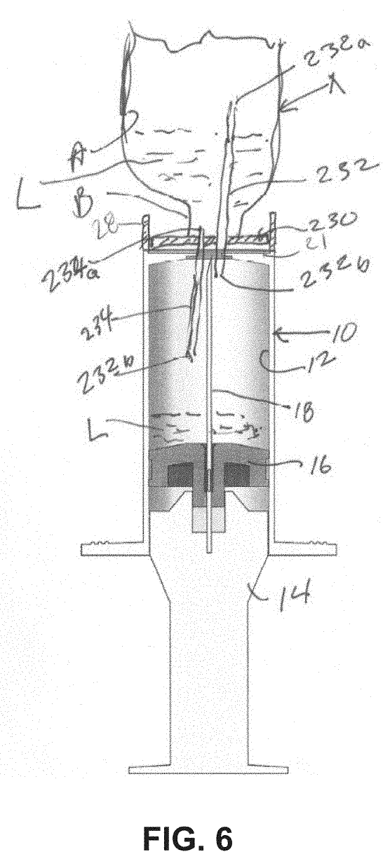

In another embodiment, as illustrated in FIG. 5 and FIG. 6, a filling adapter 230 of the vial X may comprise a sealing plate 231 generally similar to the sealing panel 34, except a first conduit 232 and a second conduit 234 may be mounted to the sealing plate 231. One or both of the first conduit 232 and the second conduit 234 may be tubular conduits. In some embodiments, the sealing plate 231 may have no septum. In some embodiments, the first conduit 232 and the second conduit 234 may be mounted to the sealing plate 231 by insertion through a septum. The first conduit 232 may be mounted to the sealing plate 231 such that a proximal end 232a of the first conduit 232 may be adapted to end a significant distance into the vial X, while a distal end 232b of the first conduit extends only a short distance beyond the sealing plate 231, as depicted in FIG. 5. The second conduit 234 may be mounted to the sealing plate 231 such that a proximal end 234a of the second conduit 234 may extend only a short distance within the vial X, while a distal end 234b of the second conduit 234 may extend a longer distance relative to the distal end 232b of the first conduit 232 within the reservoir 12 when the vial X is coupled to the syringe 1.

When the reservoir 12 of the syringe 10 is to be filled, the removable cap, cover or seal may be removed from the vial X and the filling adapter 230 may be fitted to the neck B of the vial X. As depicted in FIG. 6, the vial X may be received in the recessed cavity defined by the distal end wall 21 and the skirt 28, such that the sealing plate 231 is located within the skirt 28 extending from the distal end wall 21. The sealing plate 231 may sealingly engage with the distal end wall 21 and the skirt 28. The septum 26 of the syringe 1 may be sufficiently large, such that the septum 26 may be pierced by the first conduit 232 and the second conduit 234. When the first conduit 232 and the second conduit 234 pierces the septum 26, a portion of the first conduit 232 and the second conduit 234 may be inserted into the reservoir 12. In some embodiments, one or both of the distal ends 232b and 234b may be beveled, pointed, or otherwise shaped or formed to promote piercing of the septum 26.

In some embodiments, the syringe 1 may comprise two or more septa 26 for receiving the first conduit 232 and the second conduit 234. In such embodiments, the first conduit 232 may pierce a first septum 26, and the second conduit 234 may pierce a second septum 26.

The first conduit 232 may project a relatively short distance out from the sealing plate 231 of the filling adapter 230, and may project a relatively long distance into the vessel A, relative to the second conduit 234. When the sealing plate 231 is sealingly engaged with the syringe, such as at the distal end wall 21 and the skirt 28, the distal ends 232b and 234b may be inserted into the reservoir 12, and the proximal ends 232a and 234a may be inside the vessel A. As depicted in FIG. 6, when the vial X is engaged with the syringe 1, the liquid level of the liquid in the vial X is above the proximal end 234a of the second conduit 234 and below the proximal end 232a of the first conduit 232. The purpose of the first conduit 232 may be to displace air from the reservoir 12 into the vessel A or from the vessel A into the reservoir 12, such that the pressure in the reservoir 12 and the vessel A may approach an equilibrium. As the proximal end 234a of the second conduit 234 projects a relatively short distance into the vessel A of the vial X, the purpose of conduit 234 may be to allow liquid to flow by gravity from the vessel A into the reservoir 12. The liquid may flow from the vessel A into the reservoir 12 as the pressure in the reservoir 12 and the pressure in the vessel A approach equilibrium. In the embodiment illustrated in FIG. 5 and FIG. 6, it may not be necessary to push or pull the plunger 14 of the syringe 1, as the filling of the reservoir 12 may occur by gravity with the conduit 232 allowing the air in the reservoir 12 to be displaced into the vessel A, and the conduit 234 allowing the liquid to flow from the vessel A into the reservoir.

The adapters of the vial X for engaging the vial X with the syringe 1, such as the filling adapter 30, the bellows device 130, or the filling adapter 230, may vacuum seal the vessel A of the vial X. In some embodiments, the pressure of the vessel may be a vacuum pressure or similar to a vacuum pressure.

The adapters of the vial X for engaging the vial X with the syringe 1, such as the filling adapter 30, the bellows device 130, or the filling adapter 230, and the barrel 10, distal end wall 21, or skirt 28, may be sized or dimensioned, such that only certain vials X may be engaged with certain corresponding syringes 1. The adapter may coordinate with the syringe 1 such that only corresponding vials X may engage with the syringe 1. For example, for a 0.5 mL syringe for injecting a medicinal fluid into a child, the barrel 10 may have a particular size and dimension. The corresponding 0.5 mL vial containing the medicinal fluid for the child may have an adapter that has a similar size or dimension to engage with the 0.5 mL syringe. In this example, a 1 mL vial containing the medicinal fluid for an adult may have an adapter with different size or dimension such that the 1 mL vial may not engage with the 0.5 mL syringe. The 1 mL vial may not engage with the 0.5 mL syringe even if the vessel A of the 1 mL vial may be similar to the vessel A of the 0.5 mL vial.

In some embodiments, one or more needles may be inserted into the vial X, or one or more needles may be inserted into the reservoir 12. In some embodiments, only one vial may pierce the vial X and be inserted into the vial X.

Where the plunger 14 of the syringe 1 was pushed forward for filling the reservoir 12, such as depicted from FIG. 1 to FIG. 4, after a sufficient amount of liquid has flowed into the reservoir 12, the plunger 14 may be pulled backwards to retract the needle 18.

In some embodiments, the vial X may have a void to receive the fluid in the syringe 1, so that the fluid in the vial X may flow into the barrel 10 of the syringe 1.

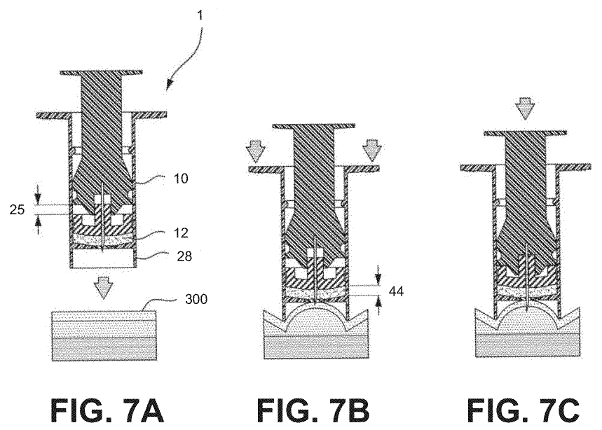

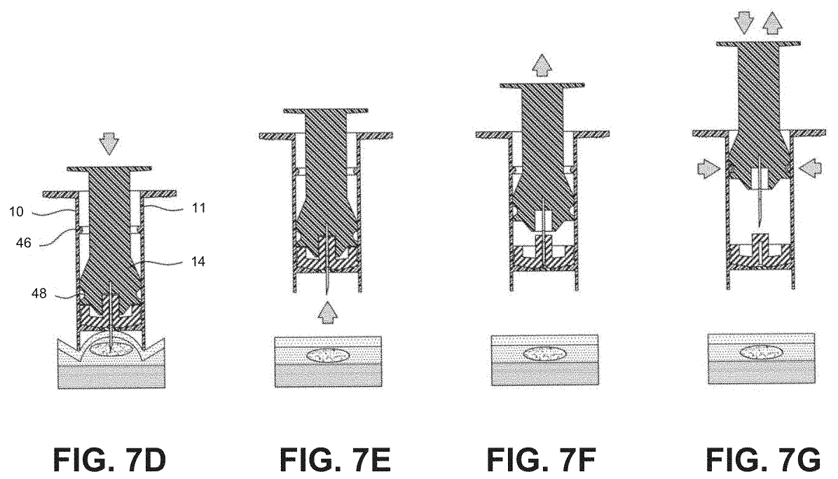

FIG. 7A to FIG. 7G illustrate an example embodiment for using the syringe 1 to inject the fluid in the barrel 10 into a patient. FIG. 7A shows the syringe 1 in an initial configuration, after the reservoir 12 has been filled with a fluid but prior to injection of the fluid. In the initial configuration, the plunger seal 16 may be positioned at an intermediate position along the longitudinal axis of the barrel 10, and the head 42 of the plunger 14 may be positioned at the proximal end 11 of the barrel 10. The chamber 24 may be defined between the plunger 14 and the plunger seal 16. The septum 26 initially closes or seals the distal end 13, and the fluid in the reservoir 12 may be trapped between the plunger seal 16, the peripheral wall of the barrel 10, and the septum 26. The plunger seal 16 may surround the needle 18 and the inlet 22. At least a portion of the passage 20 may be sufficiently large to define a recess such that the reservoir 12 and the internal conduit of the needle 18 may be in fluid communication via the inlet 22. The recess, as well as the internal conduit defined by the needle 18, may be filled with fluid in the initial configuration.

To proceed to injection, the distal end 13 of the barrel 10, and the skirt 28, are positioned against the skin 300 of the patient, as depicted in FIG. 7B. The skirt 28 may engage the skin 300 of the patient as illustrated in FIG. 7B, which may cause a puckering, or bulging, of the skin 300 as illustrated in FIG. 7B. This puckering or bulging of the skin 300 may be particularly useful in embodiments where the syringe 1 is designed for injection at subcutaneous depths.

As illustrated in FIG. 7C, a force is applied to the plunger 14 to activate the plunger 14 and to move the plunger 14 along the longitudinal axis of displacement of the barrel 10 in a forward direction, moving the anchored needle 18 with the plunger 14. The plunger 14 may first advance along the longitudinal length 25 of the chamber 24, and the free end, which may be the beveled tip 19, of the needle 18 may pierce the septum 26 and penetrate through the skin 300 and into the body of the patient. During this movement, fluid may be free to move into the recess, across the inlet 22, through the internal conduit of the needle 18, and may be injected into the patient. However, the volume of the reservoir 12 containing the fluid may remain the same during this movement, and fluid in the reservoir 12 may not be pushed across the needle inlet 22 and into the patient until the plunger 14 abuts the plunger seal 16 in the configuration shown in FIG. 7C and FIG. 7D. This may allow the needle tip 19 to reach a given penetration distance beneath the skin of the patient prior to beginning injection. The penetration distance of the needle tip 19 may be generally similar to or less than the longitudinal length 25 of the chamber 24.

In an alternate embodiment, the internal conduit defined by the needle 18 may be filled with fluid in the initial configuration, but the plunger seal 16 does not define a recess for fluid communication between the reservoir 12 and the internal conduit of the needle 18 via the inlet 22. Rather, the plunger seal 16 may abut against the needle inlet 22 and an additional portion of the needle 18 proximal or distal the needle inlet 22, such that the needle inlet 22 may be sealed by the plunger seal 16 and remains sealed by the plunger seal 16 as the plunger 14 is moved along the penetration distance. The needle inlet 22 only becomes exposed to the reservoir 12 once the plunger body 14 has been moved along the longitudinal length 25 of the channel 24 and the needle inlet 22 has crossed the plunger seal 16.

Referring now to FIG. 7C and FIG. 7D, once the plunger 14 has reached and abuts against the plunger seal 16, further movement of the plunger body 14 towards the distal end wall 21 may move both the plunger body 14 and the plunger seal 16, which, in turn, may confine the volume of the reservoir 12. The volume-confining action entrains movement of the fluid across the needle inlet 22, along the internal conduit defined by the needle 18, and out the needle tip 19 and into the patient, as the plunger 14 and plunger seal 16 are collectively moved along a longitudinal length of the reservoir 12 from the position shown in FIG. 7C to the position shown in FIG. 7D. In the position shown in FIG. 7D, the medical fluid has been injected into the patient's body.

In some embodiments, such as illustrated in FIG. 7D, the face of the plunger seal 16 that abuts the distal end wall 21 may be sized and dimensioned generally similar to the distal end wall 21. In other words, the volume-confining face of the plunger seal 16 may closely match the shape of the bottom of the barrel 10. The position of the inlet 22 may be adjusted in a manner that the inlet 22 remains exposed to the fluid until the very end of the movement (the point where the plunger seal 16 meets the septum 26 and the bottom of the barrel 10). Accordingly, the volume of fluid which remains undispensed and in the reservoir 12 after the injection operation may be minimized.

As depicted in FIG. 7E, from that position, after the fluid in the barrel 10 has been injected into the patient, the syringe 1 may be withdrawn from the patient.

As depicted in FIG. 7F, the movement of the plunger 14 may be reversed, pulling the needle 18 back into the reservoir 12. During the reversed movement, the friction between the plunger seal 16 and the barrel 10 may be greater than the friction between the plunger seal 16 and the needle 18, and the plunger seal 16 may remain in its fully deployed position at the bottom of the barrel 10 as the needle 18 is retracted. Once the inlet 22 has moved across the plunger seal 16 and is exposed to the atmosphere, the internal needle conduit may remain at atmospheric pressure.

In some embodiments, the barrel 10 may comprise a rib 46 protruding transversally inwardly near the proximal end 11 of the barrel 10, and a corresponding female feature 48, illustrated in FIG. 7A to FIG. 7G in the form of a groove or channel, provided in the head 42 of the plunger body 14. The rib 46 and the female feature 48 may be cooperatively configured for the rib 46 may be received in the female feature 48. For example, where the rib 46 has a generally round shape, the female feature 48 may also have a generally round shape. The rib 46 may extend around the entire cross-section of the barrel 10, or may extend around portions of the cross-section of the barrel 10. Similarly, the female feature 48 may extend around the entire cross-section of the plunger 14, or extend around portions of the cross-section of the plunger 14. When the plunger 14 is retracted, such as depicted in FIG. 7G, the rib 46 may engage the female feature 48, which may be a channel, provided around the head 48 of the plunger 14, which may snap the plunger 14 in the retracted position. This may prevent the plunger 14 from being retracted too far and being removed from the barrel 10.

The syringe 1 may be filled prior to being used for injection. Accordingly, the syringe 1 may be placed in storage, stocked, or transported without first filling the syringe 1. If the syringe 1 were to be damaged prior to being filled, such as by accidentally dropping the syringe and breaking the barrel 10, the fluid to be filled may be used to fill another syringe 1. Damage to syringe 1 may not also damage the fluid to be filled in the syringe 1.

The syringe 1 may comprise the needle 18 that may be retractable into the barrel 10 of the syringe 1, such that, in the retracted position, the needle 18 is entirely confined in the barrel 10. In some embodiments, the longitudinal length 29 of the skirt 28 may be longer than the longitudinal length 25 of the chamber 24. Accordingly, if the needle 18 is moved to an extended position, for example, to fill the syringe 1, such as depicted in FIG. 2, the skirt 28 may protect users from accidental nicks, pricks, or cuts as the skirt 28 may extend farther from the distal end wall 21 than the needle 18 when the needle 18 is in the extended position. In addition, while the syringe 1 is being filled by the vial X, even though the tip 19 of the needle 18 is outside the barrel 10, the tip 19 is inserted inside the vial X, such that the tip 19 is not exposed during filling of the syringe 1.

When using a vial X to fill the syringe 1, the vial X may not be pierced in the same spot twice. For example, if the vial X contains five doses of fluid to fill five syringes 1, each syringe 1 may not pierce the vial X at the same point on the septum 132, which may reduce contamination of the syringes 1 and the vial X. This may reduce errors made in the field when using the syringes 1.

When using a vial X to fill the syringe 1, the pressure in the vial X and the pressure in the barrel 10 of the syringe 1 may affect the direction of flow of the liquid. By affecting the pressure of the vial X or the pressure of the barrel 10, fluid from the vial X may flow into the barrel 10.

When using the syringe 1 for injecting a patient, the syringe 1 may be used once, such that patients may not have to worry that the needle 18 is used on more than one person.

The syringe 1 and vial X may improve safety for filling the syringe 1 with the fluid contained in vial X. For example, where the vials X has adapters that coordinate with the syringe 1 such that only corresponding vials X may engage with the syringe 1, this may reduce errors of filling the syringe 1 with the wrong fluid. For example, based on the sizes and dimensions of the syringe and the adapter of the vial, a 0.5 mL syringe for injecting a medicinal fluid into a child may not engage with a 1 mL vial containing medicinal fluid for an adult. Accordingly, a vial containing a medicinal fluid for a child may fill a syringe intended for injecting the medicinal fluid into a child.

When filling the syringe 1 with the vial X, the syringe 1 and the vial X described herein may not require vial manufacturers to redesign their vials entirely, or may not require companies that manufacture the fluid in the vials to redesign the fluid. The vessel A itself may not need to be changed. The fluid contained in the vessel A may not need to be changed. The vial X may be sealed with an adapter, such as filling adapter 30, bellows device 130, or the filling adapter 230, rather than the removable cap, cover, or seal, for engaging the vial X with the syringe 1.

When the vial X is mounted to the syringe 1 using an engagement system having a twist and lock feature, the vial X may be loaded and reloaded or otherwise mounted to different syringes. The vial X may be pierced at different positions, such that the vial X does not get pierced twice on the same septum. The needle 18 of the syringe 1 may not pierce the same septum of the vial X on more that occasion. This may reduce errors made in the field when filling and using the syringe 1.

The preceding discussion provides many example embodiments. Although each embodiment represents a single combination of inventive elements, other examples may include all suitable combinations of the disclosed elements. Thus if one embodiment comprises elements A, B, and C, and a second embodiment comprises elements B and D, other remaining combinations of A, B, C, or D, may also be used.

The term “connected” or “coupled to” may include both direct coupling (in which two elements that are coupled to each other contact each other) and indirect coupling (in which at least one additional element is located between the two elements).

Although the embodiments have been described in detail, it should be understood that various changes, substitutions and alterations can be made herein.

Moreover, the scope of the present application is not intended to be limited to the particular embodiments of the process, machine, manufacture, composition of matter, means, methods and steps described in the specification. As one of ordinary skill in the art will readily appreciate from the disclosure of the present invention, processes, machines, manufacture, compositions of matter, means, methods, or steps, presently existing or later to be developed, that perform substantially the same function or achieve substantially the same result as the corresponding embodiments described herein may be utilized. Accordingly, the appended claims are intended to include within their scope such processes, machines, manufacture, compositions of matter, means, methods, or steps.

As can be understood, the examples described above and illustrated are intended to be examples only. The invention is defined by the appended claims.

What is claimed is:

1. A syringe for receiving a fluid from a container, the syringe comprising:
a barrel for housing the fluid and including an end wall with an opening defined therein;
a plunger for slidable engagement within the barrel, and moveable between a retracted position and an extended position;
a needle having:
a first end anchored to the plunger and a second distal end;
a conduit defined by the needle; and
an inlet offset from the second distal end for fluid communication with an opening defined at the second distal end;
wherein the plunger includes a plunger seal for engagement within the barrel, the plunger seal defining a passage for receiving the needle; and
an engagement system having a complementary feature to cooperatively engage with a complementary feature of the container to mount the container to the syringe.

2. The syringe of claim 1, wherein the needle is confined in the barrel when the plunger is in the retracted position, and wherein the needle is received in the opening of the end wall when the plunger is in the extended position.

3. The syringe of claim 1, comprising a septum that seals the opening of the end wall.

4. The syringe of claim 1, comprising a skirt projecting from the end wall and defining a recessed cavity with the end wall.

5. The syringe of claim 4, wherein the skirt comprises the engagement system to mount the syringe to the container.

6. The syringe of claim 1, wherein the engagement system comprises a twist and lock feature for mounting the container to the syringe by twisting the container or syringe relative to the other.

7. The syringe of claim 1, wherein the engagement system comprises a male or female engagement part for mounting the container to the syringe by cooperatively engaging with a complementary male or female engagement part of the container.

8. The syringe of claim 1, wherein when the plunger is slidably engaged within the barrel and moved from the retracted position to the extended position, the pressure in the barrel is increased.

9. The syringe of claim 1, wherein when the container is mounted to the syringe and the plunger is in the extended position, the needle provides fluid communication between the container and the barrel for filling the barrel with fluid from the container.

10. A syringe for receiving a fluid from a container, the syringe comprising:
a barrel for housing the fluid and including an end wall with an opening defined therein;
a plunger for slidable engagement within the barrel, and moveable between a retracted position and an extended position;
a needle having:
a first end anchored to the plunger and a second distal end;
a conduit defined by the needle; and
an inlet offset from the second distal end for fluid communication with an opening defined at the second distal end;
wherein the plunger includes a plunger seal for engagement within the barrel, the plunger seal defining a passage for receiving the needle;
a chamber defined between the plunger and the plunger seal when the plunger and the plunger seal are engaged within the barrel; and
an engagement system having a complementary feature to cooperatively engage with a complementary feature of the container to mount the container to the syringe.

11. The syringe of claim 10, wherein the needle is confined in the barrel when the plunger is in the retracted position, and wherein the needle is received in the opening of the end wall when the plunger is in the extended position.

12. The syringe of claim 10, comprising a septum that seals the opening of the end wall.

13. The syringe of claim 10, comprising a skirt projecting from the end wall and defining a recessed cavity with the end wall.

14. The syringe of claim 13, wherein the skirt comprises the engagement system to mount the syringe to the container.

15. The syringe of claim 10, wherein the engagement system comprises a twist and lock feature for mounting the container to the syringe by twisting the container or syringe relative to the other.

16. The syringe of claim 10, wherein the engagement system comprises a male or female engagement part for mounting the container to the syringe by cooperatively engaging with a complementary male or female engagement part of the container.

17. The syringe of claim 10, wherein when the plunger is slidably engaged within the barrel and moved from the retracted position to the extended position, the pressure in the barrel is increased.

18. The syringe of claim 10, wherein when the container is mounted to the syringe and the plunger is in the extended position, the needle provides fluid communication between the container and the barrel for filling the barrel with fluid from the container.

19. The syringe of claim 13, wherein a longitudinal length of the skirt is greater than a longitudinal length of the chamber.

20. A syringe for receiving a fluid from a container having a first and second conduit mounted on an adapter of the container, the syringe comprising:
a barrel for housing the fluid and including an end wall with an opening defined therein;
a plunger for slidable engagement within the barrel, and moveable between a retracted position and an extended position;
a needle having:
a first end anchored to the plunger and a second distal end;
a conduit defined by the needle; and
an inlet offset from the second distal end for fluid communication with an opening defined at the second distal end;
wherein the plunger includes a plunger seal for engagement within the barrel, the plunger seal defining a passage for receiving the needle; and
an engagement system having a complementary feature to cooperatively engage with a complementary feature of the container to mount the container to the syringe;
wherein the end wall is configured to receive a first and a second conduit of the container when the container is mounted to the syringe.
